(19) United States
(12) Patent Application Publication
(10) Pub. No.: US 2009/0197899 A1
(43) Pub. Date: Aug. 6, 2009

(54) 3-(Dihydro-1H-pyrazolo[4,3-d]pyrimidin-5-yl)-4-propoxybenzenesulfonamide Derivatives and Methods of Use

(75) Inventors: Roger Tung, Lexington, MA (US); Scott Harbeson, Lexington, MA (US)

Correspondence Address: EDWARDS ANGELL PALMER & DODGE LLP, P.O. BOX 55874, BOSTON, MA, 02205, US

(73) Assignee: Concert Pharmaceuticals Inc., Lexington, MA (US)

(21) Appl. No.: 12/365,883

(22) Filed: Feb. 4, 2009

Related U.S. Application Data
(63) Continuation-in-part of application No. 12/242,869, filed on Sep. 30, 2008.
Application No. 12/242,869 is a continuation-in-part of application No. 12/112,873, filed on Apr. 30, 2008.
Application No. 12/112,873 is a continuation-in-part of application No. 11/876,754, filed on Oct. 22, 2007.
(60) Provisional application No. 61/026,130, filed on Feb. 4, 2008.
(60) Provisional application No. 60/853,234, filed on Oct. 20, 2006.

(51) Int. Cl. A61K 31/519; C07D 403/14; A61P 9/12; A61P 15/10

(52) U.S. Cl. 514/262.1; 544/262

(57) ABSTRACT

This invention relates to novel 3-(dihydro-1H-pyrazolo[4,3-d]pyrimidin-5-yl)-4-propoxybenzenesulfonamide compounds, their derivatives, pharmaceutically acceptable salts, solvates, and hydrates thereof. This invention also provides compositions comprising a compound of this invention and the use of such compositions in methods of treating diseases and conditions that are beneficially treated by administering inhibitors of cyclic guanosine 3′,5′-monophosphate specific phosphodiesterase (cGMP-specific PDE), in particular PDE5.

### CROSS-REFERENCE TO RELATED APPLICATIONS

This application is a continuation-in-part of U.S. copending patent application Ser. No. 12/242,869, filed Sep. 30, 2008, which is a continuation-in-part of copending U.S. patent application Ser. No. 12/112,873, filed Apr. 30, 2008, which is a continuation-in-part of copending U.S. patent application Ser. No. 11/876,754, filed Oct. 22, 2007, which claims the benefit of U.S. provisional patent application No. 60/853,234, filed Oct. 20, 2006. This application also claims the benefit of U.S. provisional patent application No. 61/026,130, filed Feb. 4, 2008. The contents of each of these applications are incorporated herein by reference in their entirety.

This invention relates to novel 3-(dihydro-1H-pyrazolo[4,3-d]pyrimidin-5-yl)-4-propoxybenzenesulfonamide compounds, their derivatives, pharmaceutically acceptable salts, solvates, and hydrates thereof. This invention also provides compositions comprising a compound of this invention and the use of such compositions in methods of treating diseases and conditions that are beneficially treated by administering inhibitors of cyclic guanosine 3′,5′-monophosphate specific phosphodiesterase (cGMP-specific PDE), in particular PDE5.

Udenafil, also known variously as 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide, and as 3-(1-methyl-7-oxo-3-propyl-3a,6,7,7a-tetrahydro-1H-pyrazolo[4,3-d]pyrimidin-5-yl)-N-(2-(1-methylpyrrolidin-2-yl)ethyl)-4-propoxybenzenesulfonamide, modulates activity of the cyclic guanosine monophosphate-specific phosphodiesterase type 5 (PDE5).

Udenafil has been clinically demonstrated to be an effective agent for erectile dysfunction and is currently approved for marketing in South Korea for the treatment of impotence.

Udenafil undergoes hepatic metabolism in humans, with sulfonamide N-dealkylation by cytochrome P450 3A4 (CYP3A4) being responsible for formation of the predominantly-observed circulating metabolite. Analogously with rats and dogs, additional oxidation may occur, resulting in side-chain hydroxylation and pyrazole N-dealkylation.

Side effects reportedly associated with udenafil include mild to moderate facial flushing and headache. In addition, other PDE5 inhibitors have been associated with possible vision problems.

Despite the beneficial activities of udenafil, there is a continuing need for new compounds to treat the aforementioned diseases and conditions.

### BRIEF DESCRIPTION OF THE DRAWINGS

FIG. 1 depicts metabolic stability data for compounds of the invention in human liver microsomes.

### DEFINITIONS

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein).

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of udenafil will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The term “isotopologue” refers to a species that differs from a specific compound of this invention only in the isotopic composition thereof.

The term “compound,” as used herein, when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above, the relative amount of such isotopologues in toto will be less than 55% of the compound.

The invention also provides salts of the compounds of the invention. A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula A or Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium.

“Stereoisomer” refers to both enantiomers and diastereomers.

“Tert”, “^{t}”, and “t-” each refer to tertiary.

“US” refers to the United States of America.

“FDA” refers to Food and Drug Administration.

“NDA” refers to New Drug Application.

The term “optionally substituted with deuterium” as used herein means that one or more hydrogen atoms may be replaced with a corresponding number of deuterium atoms.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula A:

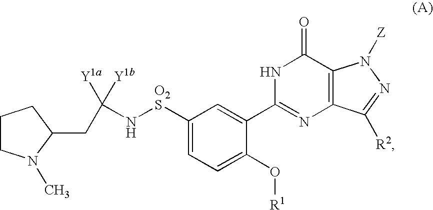

or a pharmaceutically acceptable salt thereof, wherein:

each Y¹ is independently selected from hydrogen and deuterium;

Z is selected from —CH₃, —CH₂D, —CHD₂ and —CD₃;

R¹ and R² are each independently n-propyl optionally substituted with deuterium; and

at least one of R¹, R² and Z comprises a deuterium atom, or at least one Y is deuterium.

In one embodiment, each of R¹ and R² is —(CH₂)₂CH₃, the compound having the Formula I:

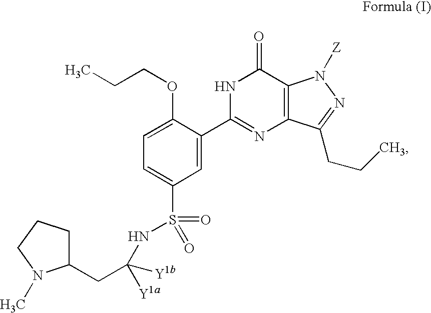

or a salt (e.g., a pharmaceutically acceptable salt) thereof, wherein each Y and Z are as defined above, and either Z comprises a deuterium or at least one Y is deuterium.

In another embodiment of Formula A, at least one of R¹ and R² comprises a deuterium atom. In one aspect of this embodiment, each of R¹ and R² is independently selected from —CH₂CH₂CH₃, —CH₂CH₂CD₃, —CD₂CH₂CH₃, —CD₂CH₂CD₃, —CH₂CD₂CH₃, —CH₂CD₂CD₃, —CD₂CD₂CH₃, and —CD₂CD₂CD₃. In a more specific aspect of this embodiment, R¹ is selected from —CH₂CH₂CH₃, and —CD₂CD₂CD₃, and R² is selected from —CH₂CH₂CH₃, —CH₂CD₂CD₃, and —CD₂CD₂CD₃. In an even more specific aspect of this embodiment, R¹ is —CD₂CD₂CD₃, and R² is —CH₂CD₂CD₃.

In another embodiment of Formula A or Formula I, Y^{1a} and Y^{1b} are the same. In one aspect of this embodiment, Y^{1a} and Y^{1b} are deuterium. In another aspect of this embodiment, Y^{1a} and Y^{1b} are hydrogen.

In another embodiment of Formula A or Formula I, Z is selected from —CH₃ and —CD₃. In one aspect of this embodiment, Z is —CH₃. In another aspect of this embodiment, Z is —CD₃.

In yet another embodiment, the compound is selected from any one of the compounds set forth in Table 1 (below):

TABLE 1
Exemplary Compounds of Formula A

| Compound | Y^{1a} | Y^{1b} | Z | R¹ | R² |
|---|---|---|---|---|---|
| 100 | D | D | CD₃ | —CH₂CH₂CH₃ | —CH₂CH₂CH₃ |
| 101 | D | D | CH₂D | —CH₂CH₂CH₃ | —CH₂CH₂CH₃ |
| 102 | D | D | CHD₂ | —CH₂CH₂CH₃ | —CH₂CH₂CH₃ |
| 103 | D | D | CH₃ | —CH₂CH₂CH₃ | —CH₂CH₂CH₃ |
| 104 | D | H | CD₃ | —CH₂CH₂CH₃ | —CH₂CH₂CH₃ |
| 105 | D | H | CH₂D | —CH₂CH₂CH₃ | —CH₂CH₂CH₃ |
| 106 | D | H | CHD₂ | —CH₂CH₂CH₃ | —CH₂CH₂CH₃ |
| 107 | D | H | CH₃ | —CH₂CH₂CH₃ | —CH₂CH₂CH₃ |
| 108 | H | H | CD₃ | —CH₂CH₂CH₃ | —CH₂CH₂CH₃ |
| 109 | H | H | CH₂D | —CH₂CH₂CH₃ | —CH₂CH₂CH₃ |
| 110 | H | H | CHD₂ | —CH₂CH₂CH₃ | —CH₂CH₂CH₃ |
| 200 | H | H | CH₃ | —CH₂CH₂CH₃ | —CH₂CD₂CD₃ |
| 201 | H | H | CH₃ | —CD₂CD₂CD₃ | —CH₂CD₂CD₃ |
| 202 | H | H | CH₃ | —CD₂CD₂CD₃ | —CH₂CH₂CH₃ |
| 203 | H | H | CD₃ | —CH₂CH₂CH₃ | —CH₂CD₂CD₃ |
| 204 | H | H | CD₃ | —CD₂CD₂CD₃ | —CH₂CD₂CD₃ |
| 205 | H | H | CD₃ | —CD₂CD₂CD₃ | —CH₂CH₂CH₃ |
| 206 | D | D | CH₃ | —CH₂CH₂CH₃ | —CH₂CD₂CD₃ |
| 207 | D | D | CH₃ | —CD₂CD₂CD₃ | —CH₂CD₂CD₃ |
| 208 | D | D | CH₃ | —CD₂CD₂CD₃ | —CH₂CH₂CH₃ |
| 209 | D | D | CD₃ | —CH₂CH₂CH₃ | —CH₂CD₂CD₃ |
| 210 | D | D | CD₃ | —CD₂CD₂CD₃ | —CH₂CD₂CD₃ |
| 211 | D | D | CD₃ | —CD₂CD₂CD₃ | —CH₂CH₂CH₃ |

In a more specific embodiment, the compound is selected from any one of Compound 100, Compound 103, Compound 108 and Compound 210

In another set of embodiments, any atom not designated as deuterium in any of the embodiments set forth above is present at its natural isotopic abundance.

The synthesis of compounds of Formula A or Formula I can be readily achieved by synthetic chemists of ordinary skill. Relevant procedures and intermediates are disclosed, for instance in U.S. Pat. Nos. 6,583,147 and 6,844,436.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

### Exemplary Synthesis

A convenient method for synthesizing 3-(dihydro-1H-pyrazolo[4,3-d]pyrimidin-5-yl)-4-propoxybenzenesulfonamide compounds of Formula A or Formula I is depicted in Scheme 1.

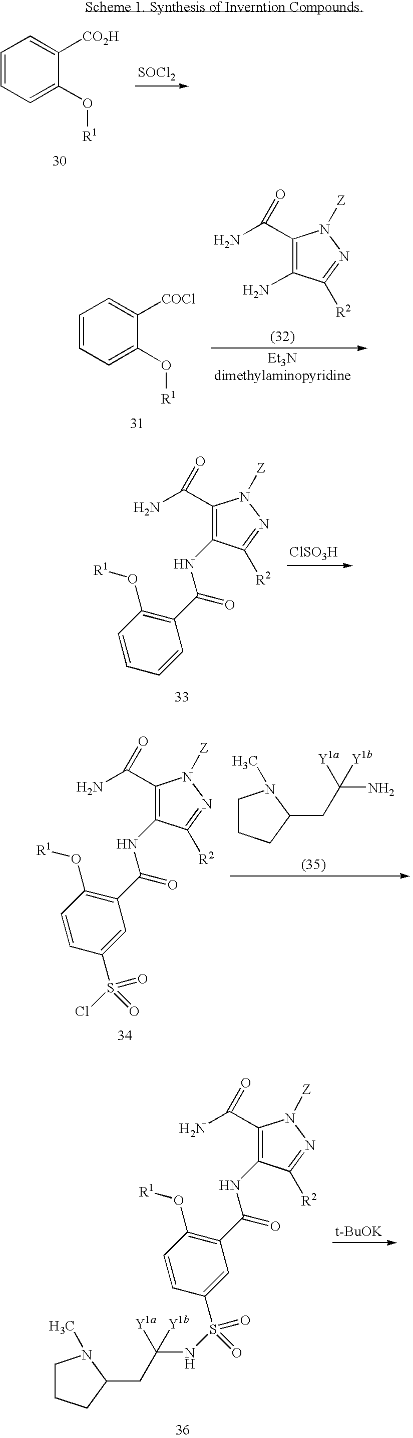

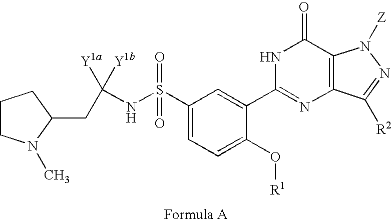

A convenient method for synthesizing compounds of Formula A is depicted in Scheme 1, above. An appropriately deuterated n-propoxybenzoic acid 30 may be treated with thionyl chloride in refluxing dichloromethane to yield the corresponding acid chloride 31, which then may be condensed with an appropriately deuterated aminopyrazole 32 in dichloromethane with triethylamine and dimethylaminopyridine to yield compound 33. Subsequent sulfonation of 33 with chlorosulfonic acid may provide the sulfonylchloride 34, which may be coupled with an appropriately substituted amine 35 in dichloromethane to yield sulfonamide 36. The desired compounds of Formula A may be obtained by cyclization of 36 in refluxing t-butanol with potassium t-butoxide.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides a pyrogen-free pharmaceutical composition comprising an effective amount of a compound of Formula A or Formula I (e.g., including any of the formulae herein), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as udenafil, i.e., any compound or therapeutic agent known to have or that demonstrates advantageous properties when administered with a PDE5 inhibitor compound. Such agents include those indicated as being useful in combination with a PDE5 inhibitor compound, or more particularly, udenafil, including but not limited to, those described in PCT publication WO2007039075 and United States patent application 20070122355.

Preferably, the second therapeutic agent is an agent useful in the treatment or prevention of a disease or condition selected from erectile dysfunction, benign prostatic hyperplasia, and pulmonary arterial hypertension, and dysmenorrhea. Exemplary second agents include angiotensin converting enzyme (ACE) inhibitors, vasopressin receptor antagonists, and angiotensin II receptor antagonists.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., 1966, Cancer Chemother. Rep, 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 50 mg to about 150 mg, or from about 10 mg to about 300 mg, or from about 5 mg to about 500 mg, or from about 1 mg to about 1000 mg.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for udenafil.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the activity of PDE5 in a cell, comprising contacting a cell with one or more compounds of Formula A or Formula I herein.

According to another embodiment, the invention provides a method of treating a patient suffering from, or susceptible to, a disease that is beneficially treated by udenafil comprising the step of administering to the patient an effective amount of a compound or a pharmaceutical composition of this invention. Such diseases are well known in the art and are disclosed in, but not limited to the following patents and published applications: PCT publication WO2004108138, WO2006132460, WO2007114534 and U.S. Pat. No. 6,583,147. Such diseases include, but are not limited to erectile dysfunction, benign prostatic hyperplasia, pulmonary arterial hypertension and dysmenorrhea.

In one particular embodiment, the method of this invention is used to treat a patient suffering from or susceptible to a disease or condition selected from erectile dysfunction, benign prostatic hyperplasia, and pulmonary arterial hypertension.

In another particular embodiment, the method of this invention is used to treat a patient suffering from or susceptible to erectile dysfunction.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to the patient one or more second therapeutic agents. The choice of second therapeutic agent may be made from any second therapeutic agent known to be useful for co-administration with a PDE5 inhibitor, more particularly udenafil. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

In particular, the combination therapies of this invention include co-administering a compound of Formula A or Formula I and a second therapeutic agent for treatment of the following conditions: erectile dysfunction, benign prostatic hyperplasia, and pulmonary arterial hypertension. In a more specific embodiment, the invention provides a method of treating dysmenorrhea by co-administering to a patient in need thereof a compound of Formula A or Formula I and a vasopressin receptor antagonist.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula A or Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula A or Formula I for use in the treatment or prevention in a patient of a disease, disorder or symptom thereof delineated herein.

### Diagnostic Methods and Kits

The compounds and compositions of this invention are also useful as reagents in methods for determining the concentration of udenafil in solution or biological sample such as plasma, examining the metabolism of udenafil and other analytical studies.

According to one embodiment, the invention provides a method of determining the concentration, in a solution or a biological sample, of udenafil, comprising the steps of:
a) adding a known concentration of a compound of Formula A or Formula I to the solution of biological sample;
b) subjecting the solution or biological sample to a measuring device that distinguishes udenafil from a compound of Formula A or Formula I;
c) calibrating the measuring device to correlate the detected quantity of the compound of Formula A or Formula I with the known concentration of the compound of Formula A or Formula I added to the biological sample or solution; and
d) measuring the quantity of udenafil in the biological sample with said calibrated measuring device; and
e) determining the concentration of udenafil in the solution of sample using the correlation between detected quantity and concentration obtained for a compound of Formula A or Formula I.

Measuring devices that can distinguish udenafil from the corresponding compound of Formula A or Formula I include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula A or Formula I comprising the steps of contacting the compound of Formula A or Formula I with a metabolizing enzyme source for a period of time and comparing the amount of the compound of Formula A or Formula I with the metabolic products of the compound of Formula A or Formula I after the period of time.

In a related embodiment, the invention provides a method of evaluating the metabolic stability of a compound of Formula A or Formula I in a patient following administration of the compound of Formula A or Formula I. This method comprises the steps of obtaining a serum, urine or feces sample from the patient at a period of time following the administration of the compound of Formula A or Formula I to the subject; and comparing the amount of the compound of Formula A or Formula I with the metabolic products of the compound of Formula A or Formula I in the serum, urine or feces sample.

The present invention also provides kits for use in treating erectile dysfunction, benign prostatic hyperplasia, and pulmonary arterial hypertension. These kits comprise (a) a pharmaceutical composition comprising a compound of Formula A or Formula I or a salt thereof, wherein said pharmaceutical composition is in a container; and (b) instructions describing a method of using the pharmaceutical composition to treat erectile dysfunction, benign prostatic hyperplasia, or pulmonary arterial hypertension.

In another embodiment, the invention provides kits for use in treating dysmenorrhea. These kits comprise (a) a pharmaceutical composition comprising a compound of Formula A or Formula I or a salt thereof, wherein said pharmaceutical composition is in a first container; (b) a pharmaceutical composition comprising a vasopressin receptor antagonist, wherein said vasopressin receptor antagonist composition is in a second container; and (c) instructions describing a method of using the pharmaceutical composition to treat dysmenorrhea.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### EXAMPLES

### Example 1

### Synthesis of Intermediate 32-d3

Intermediate 32-d3, where Z=CD₃ was prepared as outlined in Scheme 2 below. Details of the synthesis are set forth below.

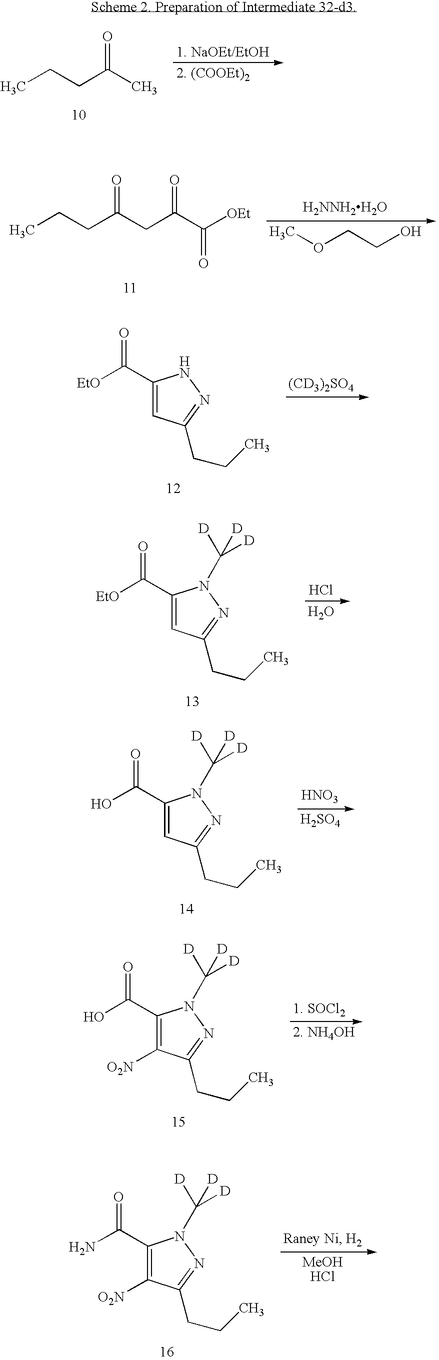

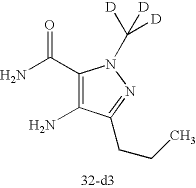

Synthesis of Ethyl 2,4-dioxoheptanoate (11). Sodium (11.5 g, 0.5 mol) was dissolved in anhydrous ethanol (180 mL) and the resulting solution was cooled to 0° C. 2-Pentanone, 10 (53 mL, 43.06 g, 0.5 mol) was added slowly. Stirring was continued at 0° C. for 20 minutes (min) and diethyl oxalate (68 mL, 73.03 g, 0.5 mol) was added drop-wise over 1.5 hours (h) at such a rate that the temperature did not exceed 5° C. Stirring was continued at 0° C. for an additional 20 min, then the mixture was allowed to warm to room temperature (rt) during which time it slowly became very viscous. The mixture was allowed to sit at rt for 36 h. Ethanol (600 mL) was added, and the reaction mixture was concentrated in vacuo. The residue was taken up in diethyl ether (400 mL), stirred on an ice bath and 2N hydrochloric acid (300 mL) was slowly added. Phases were separated and the aqueous phase was extracted with MTBE (2×400 mL). The combined organic extracts were washed with water (400 mL) and brine (400 mL), dried over sodium sulfate, filtered and the solvent was removed under reduced pressure to give ethyl 2,4-dioxoheptanoate, 11 (88.88 g, 95.5%).

Synthesis of Compound 12. A solution of ethyl 2,4-dioxoheptanoate, 11 (88.88 g, 0.4773 mol) in acetic acid (710 mL) and methoxyethanol (710 mL) was treated with hydrazine hydrate (47.8 g, 0.956 mol, 2 equiv) in 3 portions over 10 min. When the exothermal reaction subsided, the reaction mixture was stirred at 105° C. for 3.5 h. After cooling, the solvent was removed under reduced pressure (bath temperature=80-85° C.). The residue was taken up in ethyl acetate (800 mL) and water (500 mL). The phases were separated, the organic phase was washed with water (2×300 mL), dried over sodium sulfate, filtered, and the solvent was removed under reduced pressure. The crude product was purified by chromatography on silica (1.1 kg) with 8:2 heptane/ethyl acetate (18 L). Pure (48.4 g) and slightly impure 12 was obtained (85.7%).

Synthesis of Compound 13. The pyrazole carboxylate 12 (21.7 g, 0.119 mol) was slowly heated under nitrogen until it had melted completely. The temperature was increased to 140° C. and dimethyl sulfate-d₆ (ISOTEC, >99 atom % D, 7.4 g, 0.587 mol, 0.986 equiv.) was added drop-wise over 15 min. Stirring was continued at 150° C. for 3.5 h. After cooling, the reaction mixture was diluted with dichloromethane (600 mL), washed with 5% sodium carbonate (500 mL), water (500 mL) and 1:1 water/brine, dried over sodium sulfate and filtered. The solvent was removed under reduced pressure and the residue (18.1 g) was purified by chromatography on silica (350 g) with 4% ethyl acetate in dichloromethane (6 L) to give 13 (11.2 g, 47%). A small amount (1.7 g) of slightly impure product was also recovered.

Synthesis of Compound 14. A solution of 13 (11.3 g, 56.7 mmol) in 6N hydrochloric acid (125 mL) was stirred under reflux conditions overnight. After cooling, water was removed on a rotary evaporator. The residue was taken up in methanol then the solution was concentrated in vacuo. Ethyl acetate was added and again the resulting solution was concentrated in vacuo. The solid residue was dried in a vacuum oven (60° C.) to give 14 (9.51 g, 98%).

Synthesis of Compound 15. A solution of 14 (9.50 g, 55.49 mmol) in 98% sulfuric acid (40 mL) was stirred at 50° C. and a mixture of 90% nitric acid (3.6 mL) and sulfuric acid (8 mL) was added drop-wise over 10 min. Stirring was continued at this temperature for 8 h. After cooling, the reaction mixture was poured on ice (220 g) and stirred for 30 min. The precipitate was collected by filtration, washed with a little cold water, and dried in a vacuum oven to give 15 (10.24 g, 87%).

Synthesis of Compound 16. A suspension of the acid, 15 (10 g, 46.25 mmol) in thionyl chloride (30 mL) was stirred at reflux for 4 h. Excess thionyl chloride was removed under reduced pressure. Toluene was added and the resulting mixture was concentrated in vacuo. The residue was taken up in THF (15 mL) then was added slowly to ice cold 28% ammonium hydroxide with stirring. Stirring was continued at rt for 2 h. The precipitate was collected by filtration, washed with water, and dried in a vacuum oven (60° C.) to give 16 (9.72 g, 97%).

Synthesis of Intermediate 32-d3. A solution of the amide 16 (7.47 g, 34.7 mmol) in methanol (400 mL) was treated with 20% palladium on activated carbon (50% wet, 0.5 g) and 4N hydrochloric acid in dioxane (30 mL, 120 mmol) then was hydrogenated at 50 psi for 2 h. The catalyst was filtered off and washed with methanol. The solvent was removed in vacuo and the residue was dried in a vacuum oven (60° C.) to give intermediate 32-d3 as the hydrochloride salt (7.57 g, 98%).

### Example 2

### Synthesis of Intermediate 35-d2

Intermediate 35-d2, where Y^{1a,1b}=D, was prepared as outlined in Scheme 3 below. Details of the synthesis are set forth below.

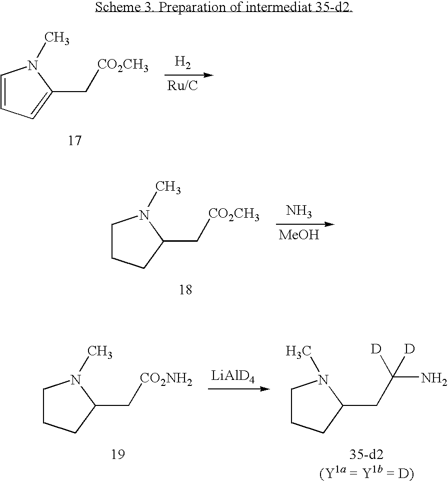

Synthesis of Compound 18. A solution of methyl 1-methylpyrrole acetate 17 (18.9 g, 0.123 mol) in methanol (800 mL) was treated with 5% rhodium on activated carbon and hydrogenated at 40 psi for 30 h. The catalyst was filtered off and washed with methanol. The solvent was removed under reduced pressure and the crude product was purified by chromatography on silica (250 g) with 2% 7N methanolic ammonia in dichloromethane (2 L) and 4% 7N methanolic ammonia in dichloromethane (1.5 L) to give 18 (16.4 g, 85%).

Synthesis of Compound 19. A solution of 18 (17.17 g, 10.92 mmol) in 7N ammonia in methanol (800 mL) was stirred in a Parr pressure reactor at 125° C. overnight. After cooling the resultant solution was rid of solvent under reduced pressure to give 19 (11.3 g, 77%).

Synthesis of Intermediate 35-d2 (Y^{1a}═Y^{1b}=D). A suspension of lithium aluminum deuteride (Cambridge Isotopes, 98 atom % D, 6.42 g, 152.8 mmol) in anhydrous tetrahydrofuran (160 mL) was stirred under nitrogen at 0° C. and a solution of 19 (10.8 g, 75.9 mmol) in anhydrous tetrahydrofuran (210 mL) was added at such a rate that the temperature did not exceed 5° C. After complete addition, stirring was continued at 0° C. for 20 min, then at rt for 1 h and finally at 60° C. for 3 h. The resultant mixture was cooled to 0° C. and water (6.4 mL) was added very carefully, followed by 10% sodium hydroxide solution (6.4 mL) and water (19.2 mL). The resulting suspension was stirred at rt overnight, filtered through celite, and the filter cake was washed with tetrahydrofuran. The solvent was removed in vacuo and the residue was vacuum distilled. The fraction distilling at 54-59° C./6 mm was collected to give 35-d2 (Y^{1a} Y^{1b}=D), (7.25 g, 73.3%).

### Example 3

### Synthesis of Compound 103

Compound 103 was prepared in a manner similar to that shown in Scheme 1 above using appropriately deuterated solvents. Details of the synthesis are set forth below.

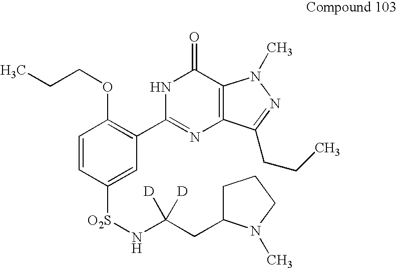

Synthesis of Methyl 2-propoxybenzoate. A mixture of methyl salicylate (8.4 g, 55 mmol), propargyl bromide (6.15 g, 50 mol) and potassium carbonate (138 g, 100 mmol) in anhydrous acetonitrile (100 mL) was kept at reflux under nitrogen with stirring overnight. After cooling the reaction mixture was filtered through celite and the filter cake was washed with acetonitrile. The solvent was removed in vacuo. The residue was taken up in ethyl acetate (150 mL), washed with water (2×100 mL), dried over sodium sulfate and filtered. The solvent was removed in vacuo and the crude product was purified by chromatography on silica (180 g) with 2.5% ethyl acetate in heptane (2 L) and 5% ethyl acetate in heptane (2 L) to give methyl 2-propoxybenzoate (7.4 g, 76.3%).

Synthesis of Compound 30. A solution of methyl 2-propoxybenzoate (5.72 g, 29.4 mmol) in tetrahydrofuran (90 mL) and 10% sodium hydroxide (45 mL) was stirred at reflux for 5 h. Tetrahydrofuran was removed under reduced pressure and the aqueous residue was washed with MTBE (2×80 mL), cooled on an ice bath and made acidic with 6N hydrochloric acid. The resulting mixture was extracted with ethyl acetate (2×200 mL). The combined extracts were dried over sodium sulfate and filtered. The solvent was removed under reduced pressure to give Ia (5.29 g, 100%).

Synthesis of Compound 33 (Z=CH₃). A solution of 30 (5.21 g, 28.9 mmol) in dichloromethane (30 mL) was treated with thionyl chloride (13.75 g, 115.5 mmol) then stirred under reflux conditions for 3.5 h. Solvent and excess thionyl chloride were removed on a rotary evaporator. The crude acid chloride 31 was taken in dichloromethane (30 mL) and added at 0° C. to a suspension of 32 (Z=CH₃) (5 g, 27.4 mmol) in dichloromethane (50 mL). This mixture was stirred on the ice bath and triethylamine (2.8 g, 27.7 mmol) in dichloromethane (15 mL) was added at such a rate that the temperature did not exceed 5° C. Stirring was continued at 0° C. for 15 min and at rt for 1.5 h. The reaction mixture was diluted with dichloromethane (150 mL), washed with water (2×80 mL), saturated sodium bicarbonate solution (80 mL) and brine (80 mL), dried over sodium sulfate and filtered. The solvent was removed on a rotary evaporator. The residue was taken up in heptane (350 mL), stirred at 70° C. for 20 min and allowed to cool overnight. The precipitate was collected by filtration, washed with heptane and dried in a vacuum oven (65° C.) to give 33 (Z=CH₃), (8.26 g, 87.4%).

Synthesis of Compound 34 (Z=CH₃). Compound 33 above (8.12 g, 23.6 mmol) was slowly added to chlorosulfonic acid (20 mL) with stirring on an ice bath under nitrogen at such a rate that the temperature did not exceed 5° C. After complete addition, stirring was continued on the ice bath for 15 min and at rt overnight. The reaction mixture was poured on ice, water was added (to bring the volume to 180 mL), and the resulting suspension was stirred at rt for 1 h. The precipitate was collected by filtration, washed with water, dissolved in ethyl acetate (700 mL), washed with water (2×200 mL) and brine (200 mL), dried over sodium sulfate, and filtered. The solvent was removed under reduced pressure and the product was dried in a vacuum oven (60° C.) to give 34 (Z=CH₃), (8.6 g, 82.3%).

Synthesis of Compound 36-d2 (Y^{1a}═Y^{1b}=D, Z=CH₃). A solution of intermediate 34 above (2.43 g, 5.48 mmol) in dichloromethane (35 mL) was stirred under nitrogen on an ice bath and 35-d2 (Y^{1a}═Y^{1b}=D, see Example 2, above) (1.5 g, 2 equiv.) was added. Stirring was continued at rt for 1 h. The reaction mixture was diluted with dichloromethane (to 180 mL), washed with water (2×100 mL), saturated sodium bicarbonate solution (100 mL), water (100 mL) and brine (100 mL), dried over sodium sulfate and filtered. The solvent was removed under reduced pressure, the crude product (2.72 g) was taken up in hot ethyl acetate (100 mL, 60° C.), and the volume was reduced to 25 mL. The resulting solution was allowed to cool with stirring overnight. The precipitate was collected by filtration, washed with a little cold ethyl acetate and dried in a vacuum oven (60° C.) to give 36-d2 (Y^{1a}═Y^{1b}=D, Z=CH₃), (2.25 g, 76.5%).

Synthesis of Compound 103. A solution of 36-d2 above (2.1 g, 3.91 mmol) and potassium tert-butoxide (0.88 g, 7.84 mmol) in tert-butanol (35 mL) was stirred at reflux under nitrogen for 5 h. After cooling, the reaction mixture was diluted with ethyl acetate (200 mL), washed with water (2×150 mL) and brine (150 mL), dried over sodium sulfate, filtered and the solvent was removed under reduced pressure. The crude product was taken up in ethyl acetate (80 mL), treated with charcoal, stirred at 50° C. for 20 min and filtered. The solvent was removed under reduced pressure and the residue was dried in a vacuum oven (60° C.) to give 103 (1.8 g, 89%). ¹H-NMR (300 MHz, CDCl₃): δ 1.04 (t, J=7.3, 3H), 1.19 (t, J=7.2, 3H), 1.39-1.64 (m, 5H), 1.70-1.89 (m, 4H), 2.01-2.16 (m, 3H), 2.31 (s, 3H), 2.37-2.41 (m, 1H), 2.91 (dd, J₁=7.9, J₂=5.9, 2H), 3.03-3.10 (m, 1H), 4.24-4.28 (m, 5H), 7.14 (d, J=8.8, 1H), 7.95 (dd, J₁=8.8, J₂=2.3, 1H), 8.93 (d, J=2.3, 1H), 10.92 (bs, 1H). ¹³C-NMR (75 MHz, CDCl₃): δ 10.65, 14.05, 22.36, 22.40, 22.63, 27.64, 27.83, 27.92, 38.24, 40.69, 56.84, 64.83, 71.81, 112.82, 120.97, 124.48, 130.25, 131.20, 133.64, 138.35, 146.66, 146.94, 153.61, 159.04. HPLC (method: 20 mm C18-RP column-gradient method 2-95% ACN+0.1% formic acid in 3.3 min with 1.7 min hold at 95% ACN; Wavelength: 254 nm): retention time: 2.72 min; 99.2% purity. MS (M+H): 519.3. Elemental Analysis (C₂₅H₃₄D₂N₆O₄S): Calculated: C=57.89, H=7.00, N=16.20, S=6.18. Found: C=57.59, H=7.07, N=15.99, S=6.18.

### Example 4

### Synthesis of Compound 108

Compound 108 was prepared in a manner similar to that shown in Scheme 1 above using appropriately deuterated reagents. Details of the synthesis are set forth below.

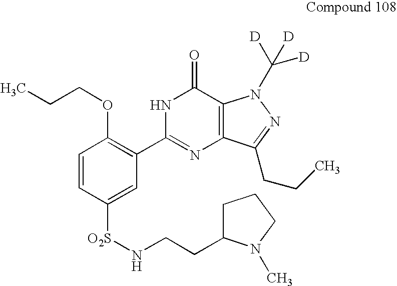

Synthesis of 33-d3 (Z=CD₃). A solution of 30 (prepared as described in Example 3; 5.8 g, 32.2 mmol) in dichloromethane (60 mL) was treated with thionyl chloride (15.2 g, 9.3 mmol) and stirred under reflux conditions for 3.5 h. Solvent and excess thionyl chloride were removed on a rotary evaporator. The crude acid chloride (31) was taken up in dichloromethane (15 mL) and added at 0° C. to a suspension of 32 (6.8 g, 30.7 mmol) in dichloromethane (40 mL). This mixture was stirred at 0° C. and triethylamine (7.6 g, 75.2 mmol) in dichloromethane (15 mL) was added at such a rate that the temperature did not exceed 5° C. Stirring was continued at 0° C. for 15 min and at rt for 1 h. The reaction mixture was diluted with dichloromethane (150 mL), washed with water (2×200 mL) and saturated sodium bicarbonate solution (200 mL), dried over sodium sulfate, filtered and the solvent was removed under reduced pressure. The residue was taken up in heptane (350 mL), stirred at 70° C. for 20 min and allowed to cool overnight. The precipitate was collected by filtration, washed with heptane and dried in a vacuum oven (60° C.) to give 33-d3 (Z=CD₃) (10.0 g, 93.8%).

Synthesis of Compound 34-d3 (Z=CD₃). Chlorosulfonic acid (15 mL) was stirred on an ice bath under nitrogen then, intermediate 33-d3 above (5 g, 14.4 mmol) was slowly added at such a rate that the temperature did not exceed 5° C. After complete addition stirring was continued on the ice bath for 15 min and at rt overnight. The reaction mixture was poured on ice. Water was added (to 200 mL) and the resulting suspension was stirred at rt for 1 h. The precipitate was collected by filtration, washed with water, dissolved in ethyl acetate, washed with water (2×150 mL) and brine (150 mL), dried over sodium sulfate and filtered. The solvent was removed in a rotary evaporator to give Compound 34-d3 (Z=CD₃), (5.5 g, 85%).

Synthesis of Compound 36-d3 (Y^{1a}═Y^{1b}═H, Z=CD₃). A solution of Compound 34-d3 above (0.8 g, 1.8 mmol) in dichloromethane (10 mL) was stirred under nitrogen on an ice bath and 35 (Y^{1a}═Y^{1b}═H) (commercial, 0.5 g, 3.8 mmol) in dichloromethane (5 mL) was added at such a rate that the temperature did not exceed 5° C. Stirring was continued at rt for 1 h. The reaction mixture was diluted with dichloromethane (to 150 mL), washed with water (2×80 mL), saturated sodium bicarbonate solution (80 mL) and brine (80 mL), dried over sodium sulfate, filtered and the solvent was removed under reduced pressure. The solid residue was dissolved in ethyl acetate (30 mL) at 60° C. (water bath), the volume was reduced to 8 mL and this solution was allowed to sit overnight. The precipitate was collected by filtration, washed with cold ethyl acetate and dried in a vacuum oven (60° C.) to give Compound 36-d3 (Y^{1a}═Y^{1b}═H, Z=CD₃), (0.80 g, 93%).

Synthesis of Compound 108. A solution of Compound 36-d3 above (0.80 g, 1.49 mmol) and potassium tert-butoxide (0.34 g, 3 mmol) in tert-butanol (25 mL) was stirred at reflux under nitrogen for 5 h. After cooling, the reaction mixture was diluted with ethyl acetate (to 150 mL), washed with water (2×100 mL) and brine (100 mL), dried over sodium sulfate and filtered. The solvent was removed under reduced pressure and the crude product (0.75 g) was dissolved in hot ethyl acetate (55° C.), treated with charcoal and filtered hot. The solvent was removed under reduced pressure and the product was dried in a vacuum oven (60° C.) to give Compound 108 (0.69 g, 89%). ¹H-NMR (300 MHz, CDCl₃): δ 1.03 (t, J=7.5, 3H), 1.19 (t, J=7.5, 3H), 1.39-1.64 (m, 4H), 1.71-1.91 (m, 4H), 1.99-2.18 (m, 3H), 2.31 (s, 3H), 2.33-2.42 (m, 1H), 2.90-2.95 (m, 2H), 3.04-3.15 (m, 3H), 4.26 (t, J=6.4, 2H), 7.14 (d, J=8.8, 1H), 7.93 (dd, J₁=8.8, J₂=2.6, 1H), 8.92 (d, J=2.6, 1H). ¹³C-NMR (75 MHz, CDCl₃): δ 10.64, 14.05, 22.36, 22.39, 22.64, 27.64, 27.95, 28.10, 40.29, 40.69, 56.84, 64.86, 71.81, 112.832, 120.99, 124.49, 130.26, 131.21, 133.59, 138.35, 146.67, 146.94, 153.621, 159.04. HPLC (method: 20 mm C18-RP column-gradient method 2-95% ACN+0.1% formic acid in 3.3 min with 1.7 min hold at 95% ACN; Wavelength: 254 nm): retention time: 2.72 min; 99.6% purity. MS (M+H): 520.2. Elemental Analysis (C₂₅H₃₃D₃N₆O₄S): Calculated: C=57.78, H=6.98, N=16.17, S=6.17. Found: C=57.50, H=7.08, N=15.77, S=6.15.

### Example 5

### Synthesis of Compound 100

Compound 100 was prepared in a manner similar to that shown in Scheme 1 above using appropriately deuterated solvents. Details of the synthesis are set forth below.

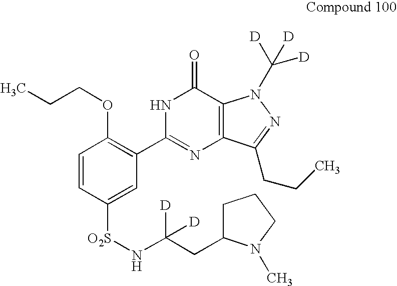

Synthesis of Compound 36-d5 (Y^{1a}═Y^{1b}=D, Z=CD₃). A solution of Compound 34-d3 (Z=CD₃, see Example 4) (1.6 g, 3.59 mmol) in dichloromethane (25 mL) was stirred under nitrogen at 0° C. and 35-d2 (Y^{1a}═Y^{1b}=D, see Example 2) (0.94 g, 7.18 mmol, 2 equiv.) in dichloromethane (10 mL) was slowly added. Stirring was continued at rt for 1 h. The reaction mixture was diluted with dichloromethane (to 150 mL), washed with water (2×100 mL), saturated sodium bicarbonate solution (100 mL), water (100 mL) and brine (100 mL), dried over sodium sulfate, filtered and the solvent was removed under reduced pressure. The crude product was taken up in hot ethyl acetate (50 mL, 60° C.) and the volume was reduced to 10 mL. The resulting solution was allowed to cool overnight. The precipitate was collected by filtration, washed with cold ethyl acetate and dried in a vacuum oven (60° C.) to give 36-d5 (Y^{1a}═Y^{1b}=D, Z=CD₃), (1.39 g, 72%).

Synthesis of Compound 100. A solution of 36-d5 above (1.39 g, 2.58 mmol) and potassium tert-butoxide (0.56 g, 5 mmol) in tert-butanol (45 mL) was stirred at reflux under nitrogen for 5 h. After cooling the reaction mixture was diluted with ethyl acetate (150 mL), washed with water (2×80 mL) and brine (80 mL), dried over sodium sulfate, filtered and the solvent was removed under reduced pressure. The crude product was taken up in ethyl acetate (50 mL), treated with charcoal, stirred hot at 50° C. for 20 min and filtered. The solvent was removed under reduced pressure and the residue was dried in a vacuum oven (60° C.) to give 100 (1.12 g, 84%). ¹H-NMR (300 MHz, CDCl₃): δ 1.03 (t, J=7.3, 3H), 1.19 (t, J=7.3, 3H), 1.43-1.62 (m, 4H), 1.73-1.89 (m, 4H), 2.01-2.16 (m, 3H), 2.31 (s, 3H), 2.38-2.41 (m, 1H), 2.90-2.95 (m, 2H), 3.06-3.09 (m, 1H), 4.26 (t, J=6.4, 5H), 7.14 (d, J=8.8, 1H), 7.95 (dd, J₁=8.8, J₂=2.3, 1H), 8.93 (d, J=2.3, 1H). ¹³C-NMR (75 MHz, CDCl₃): δ 10.64, 14.05, 22.36, 22.40, 22.63, 27.64, 27.85, 27.93, 40.69, 56.84, 64.84, 71.81, 112.83, 120.98, 124.50, 130.26, 131.20, 133.66, 138.35, 146.65, 146.95, 153.61, 159.04. HPLC (method: 20 mm C18-RP column-gradient method 2-95% ACN+0.1% formic acid in 3.3 min with 1.7 min hold at 95% ACN; Wavelength: 254 nm): retention time: 2.72 min; 99.6% purity. MS (M+H): 522.3. Elemental Analysis (C₂₅H₃₁D₅N₆O₄S): Calculated: C=57.56, H=6.96, N=16.11, S=6.15. Found: C=57.25, H=6.90, N=15.76, S=6.20.

### Example 6

### Synthesis of Intermediate 30-d7

Intermediate 30-d7, where R¹=CD₂CD₂CD₃, is prepared as outlined in Scheme 4 below and as described below.

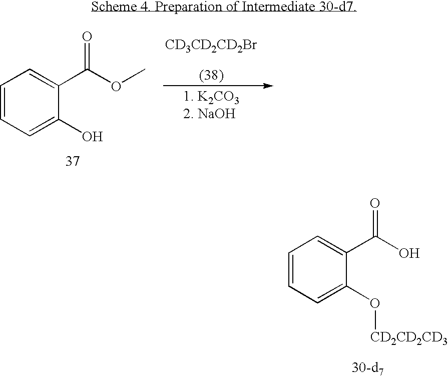

Intermediate 30-d₇ is prepared from commercially available methyl salicylate (37) by alkylation of the phenol with d₇-1-bromopropane 38, followed by saponification of the methyl ester with aqueous base (Coates, W J et al., J Med Chem, 1993, 36(10): 1387-1392).

### Example 7a

### Synthesis of 32-d5

Intermediate 32-d5, where Z=CH₃, and R²═CH₂CD₂CD₃, is prepared as outlined in Scheme 5 below, and as described below.

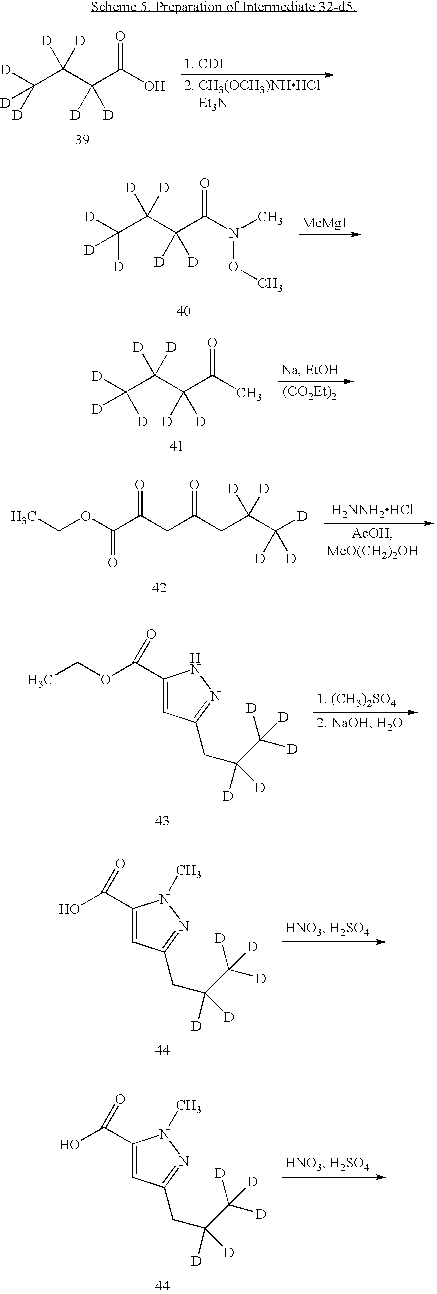

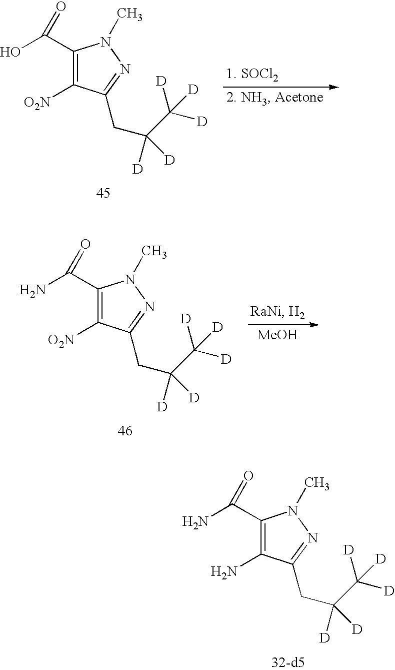

4-Amino-1-methyl-3-(2,2,3,3,3-d₅-propyl)-1H-pyrazole-5-carboxamide 32-d5 is prepared from commercially available d₇-butyric acid (39), which is activated with carbonyl diimidazole (CDI) for coupling with N-methyl-O-methylhydroxylamine HCl to provide the Weinreb amide 40. The Weinreb amide 40 is converted to the methyl ketone 41 with methyl Grignard followed by condensation with diethyloxylate to yield dioxoheptanoate 42. Cyclization of 42 with hydrazine provides the pyrazole 43, which is then methylated with dimethylsulfate to the methyl pyrazole and then saponified to the free acid 44. Nitration of 44 yields nitrate 45, which is then activated with thionyl chloride to provide the acid chloride, which is subsequently reacted with ammonia to provide the amide 46. Reduction of 46 with Raney nickel yields 32-d5, wherein R² is —CH₂CD₂CD₃ (Hanning, H et al., Bioorg Med Chem Let, 2005, 15(17): 3900-3907, and PCT Intl publication WO2003053974).

### Example 7b

### Synthesis of 32-d7

Intermediate 32-d7, where Z=CH₃, and R²═CD₂CD₂CD₃, is prepared as outlined in Scheme 5 above, using EtOD in the conversion of methyl ketone 41 to dioxoheptanoate 42, followed by the use of deuterated reagents in the conversion of 42 to 43 to allow for production of 32-d7.

### Example 8

### Synthesis of 4-Amino-1-(methyl-d₃)-3-(2,2,3,3,3-d₅-propyl)-1H-pyrazole-5-carboxamide Hydrochloride (32-d8)

Intermediate 32-d8 was prepared as outlined in Scheme 2, above, using appropriately deuterated intermediates, and starting with intermediate 10-d7, prepared as outlined in Scheme 6, below.

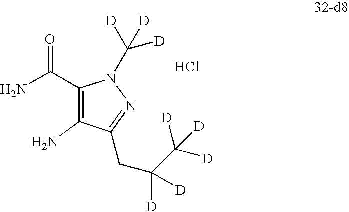

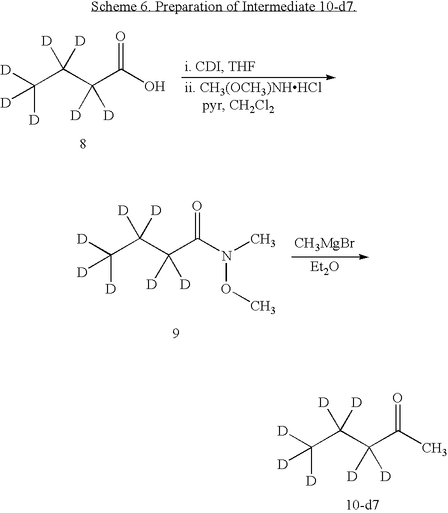

Synthesis of N-Methoxy-N-methyl(d₇-butyramide) (9). Carbonyl diimidazole (CDI, 42.8 g, 0.264 mol) was suspended in anhydrous tetrahydrofuran (250 mL) under nitrogen and d7-butyric acid 8 (Aldrich, 98 atom % D, 25 g, 0.263 mol) was added in 5 g portions. Stirring was continued at rt for 1.5 h. A solution of pyridine (28 g, 0.354 mol, 1.35 equiv.) in dichloromethane (250 mL) was stirred under nitrogen on an ice bath and N,O-dimethyl hydroxylamine hydrochloride (27.4 g, 0.28 mol, 1.07 equiv.) was added. This mixture was stirred on the ice bath for 1 h, while the tetrahydrofuran solution of d7-butyric imidazolide was slowly added at such a rate that the temperature did not exceed 5° C. After complete addition, stirring was continued at rt for 2 h. The reaction mixture was diluted with dichloromethane (to 1.5 L) and washed with water (750 mL), 1N hydrochloric acid (2×500 mL), water (750 mL) and brine (750 mL), dried over sodium sulfate and filtered. The solvent was removed in vacuo (bath temperature<22° C.) to give a mixture of the product 9 (36 g, 99%) and dichloromethane (13 g), as assessed by proton NMR.

Synthesis of 3,3,4,4,5,5,5-d₇-Pentan-2-one (10-d7). A solution of 9 (72 g, 0.521 mol) in diethyl ether (800 mL) was stirred under nitrogen on an ice bath and 3M methyl magnesium bromide (260 mL, 1.5 equiv.) was added at such a rate that the temperature did not exceed 5° C. Stirring was continued for 2 h. The reaction mixture was slowly added at ice bath temperature to saturated ammonium chloride solution (200 mL) with good stirring. Stirring was continued for 30 min. Phases were separated and the aqueous layer was extracted with diethyl ether (3×400 mL). The combined organic extracts were washed with brine (500 mL), dried over sodium sulfate and filtered. Ethylene glycol (70 g) was added and this mixture was distilled at atmospheric pressure. The product distilled at 94-104° C., partially as an azeotrope with water. The water, which separated, was drained off and the clear product was dried over sodium sulfate to give 10-d7 (38.4 g, 79%).

Synthesis of Ethyl 2,4-Dioxo-6,6,7,7,7-d₅-heptanoate (11-d5). Sodium (9.5 g, 0.413 mol) was added to anhydrous ethanol (300 mL) and stirred until all sodium had dissolved. The resulting solution was cooled on an ice bath and 10-d7 (38.4 g, 0.412 mol) was added over 20 min. Stirring was continued on the ice bath for 20 min and diethyl oxalate (60.2 g, 0.412 mol) was added over 20 min. Stirring was continued on the ice bath for 20 min and then at rt overnight. The solvent was removed in vacuo. The residue was taken up in MTBE (400 mL), cooled on an ice bath and 2N hydrochloric acid (300 mL) was added with stirring. The phases were separated and the aqueous phase was extracted with MTBE (2×400 mL). The combined organic extracts were washed with water (400 mL) and brine (400 mL), dried over sodium sulfate and filtered. The solvent was removed in vacuo to give 11-d5 (64.95 g, 82.3%).

Synthesis of Ethyl 3-(2,2,3,3,3-d₅-Propyl)-1H-pyrazole-5-carboxylate (12-d5). A solution of 11-d5 (40.14 g, 0.21 mol) in acetic acid (300 mL) and methoxyethanol (300 mL) was stirred under nitrogen at rt and hydrazine hydrate (21 g, 0.42 mol, 2 equiv.) was slowly added. The temperature reached 54° C. When the exothermal reaction subsided the reaction mixture was stirred at 105° C. for 3.5 h. The solvent was removed in vacuo and the residue was taken up in ethyl acetate (800 mL) and water (400 mL). This mixture was stirred well and sodium bicarbonate was slowly added until the aqueous phase reached pH=7.5. The phases were separated. The aqueous phase was extracted with ethyl acetate (400 mL) and the combined organic extracts were washed with water (2×400 mL), dried over sodium sulfate and filtered. The solvent was removed in vacuo and the residue (39 g) was purified by chromatography on silica (800 g) with dichloromethane (6 L), 5% ethyl acetate in dichloromethane (8 L) and 10% ethyl acetate in dichloromethane (12 L) to give 12-d5 (26.7 g, 68%). An additional 4.8 g of lesser quality product was also obtained.

Synthesis of Ethyl 1-(Methyl-d₃)-3-(2,2,3,3,3-d₅-propyl)-1H-pyrazole-5-carboxylate (13-d8). A mixture of 12-d5 (26 g, 0.139 mol) and dimethyl sulfate-d6 (ISOTEC, >99 atom % D, 10 g, 0.079 mol) was stirred at rt until the resulting exothermal reaction subsided, then at 160° C. for 5 h. After cooling, the reaction mixture was taken up in dichloromethane (600 mL), washed with 5% sodium carbonate (500 mL) and water (2×400 mL), dried over sodium sulfate and filtered. The solvent was removed in vacuo and the residue (4.2 g) was purified by chromatography on silica (145 g) with 9:1 heptane/ethyl acetate (2 L) to give 13-d8 (3.4 g, 12%).

Synthesis of 1-(Methyl-d₃)-3-(2,2,3,3,3-d₅-propyl)-1H-pyrazole-5-carboxylic acid (14-d8). A solution of 13-d8 (3.4 g, 16.64 mmol) in 6N hydrochloric acid (45 mL) was stirred at reflux overnight. After cooling, water was removed in vacuo. Methanol (60 mL) was added and removed in vacuo. Ethyl acetate (2×60 mL) was added and removed in vacuo. The residue was dried in a vacuum oven (60° C.) to give 14-d8 (2.87 g, 98%).

Synthesis of 1-(Methyl-d₃)-4-nitro-3-(2,2,3,3,3-d₅-propyl)-1H-pyrazole-5-carboxylic acid (15-d8). A solution of 14-d8 (2.87 g, 16.28 mmol) in concentrated sulfuric acid (12 mL) was stirred under nitrogen at 50° C. and a mixture of 90% nitric acid (1.40 g) and sulfuric acid (3 mL) was slowly added. The resulting reaction mixture was stirred under nitrogen at 50° C. for 8 h. After cooling, the reaction mixture was poured on ice. Water was added (to 150 mL) and the resulting suspension was stirred for 30 min. The precipitate was collected by filtration, washed with a little cold water and dried in a vacuum oven (60° C.) to give 15-d8 (2.84 g, 79%).

Synthesis of 1-(Methyl-d₃)-4-nitro-3-(2,2,3,3,3-d₅-propyl)-1H-pyrazole-5-carboxamide (16-d8). A solution of 15-d8 (2.7 g, 12.2 mmol) in thionyl chloride (20 mL) was stirred at reflux for 4 h. Excess thionyl chloride was removed in vacuo. The residue was taken up in tetrahydrofuran (5 mL) and added slowly to 27% ammonium hydroxide at 0° C. Stirring was continued on the ice bath for 30 min and the resulting suspension was allowed to sit overnight at rt. The precipitate was collected by filtration, washed with water and dried in a vacuum oven (60° C.) to give 16-d8 (2.41 g, 89.6%).

Synthesis of 4-Amino-1-(methyl-d₃)-3-(2,2,3,3,3-d₅-propyl)-1H-pyrazole-5-carboxamide hydrochloride (32-d8). A solution of 16-d8 (2.2 g, 10 mmol) in methanol (40 mL) and 4N hydrogen chloride in dioxane (20 mL, 80 mmol) was treated with 20% palladium on activated carbon (50% wet, 0.2 g). The reaction mixture was subjected to hydrogenation at 50 psi for 4 h, after which time a heavy white precipitate had formed. Additional methanol (80 mL) and 20% palladium on activated carbon (50% wet, 0.2 g) were added and hydrogenation at 50 psi was continued for an additional 2 h. The catalyst was removed by filtration and washed with methanol. The solvent was removed in vacuo and the residue was dried in a vacuum oven (65° C.) to give 32-d8 (2.3 g, 100%).

### Example 9

### Synthesis of 3-(1-(Methyl-d₃)-7-oxo-3-(2,2,3,3,3-d₅-propyl)-6,7-dihydro-1H-pyrazolo[4,3-d]pyrimidin-5-yl)-N-(2-(1-methylpyrrolidin-2-yl)-1,1-d₂-ethyl)-4-(propoxy-d₇)-benzenesulfonamide (Compound 210)

Compound 210 was prepared as outlined in Scheme 1, above, using appropriately deuterated intermediates, and starting with intermediate 30-d7, prepared as outlined in Scheme 7, below.

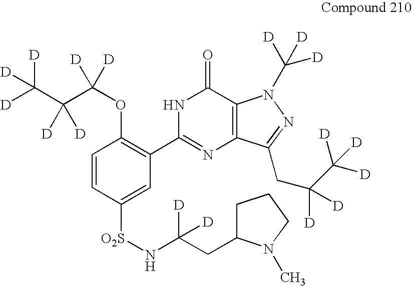

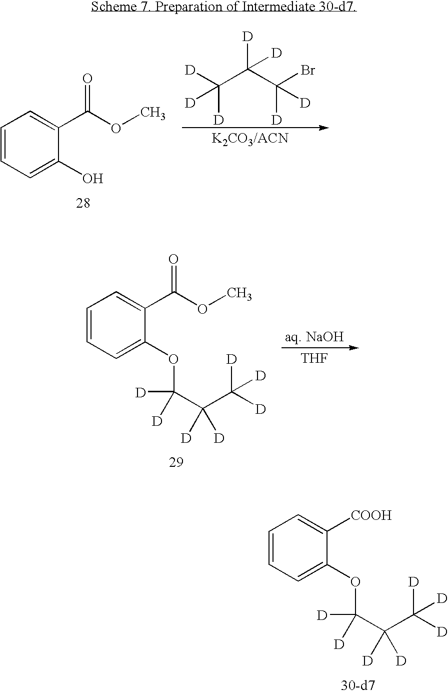

Synthesis of Methyl 2-(Propoxy-d₇)benzoate (29). A mixture of methyl salicylate 28 (5 g, 32.86 mmol), 1-bromopropane-d7 (ISOTEC, >98 atom % D, 3.9 g, 30 mmol) and potassium carbonate (8.3 g, 60 mmol) in anhydrous acetonitrile (60 mL) under nitrogen was stirred at reflux overnight. After cooling, the reaction mixture was diluted with acetonitrile (80 mL), filtered through Celite and the filter cake was washed with acetonitrile. The solvent was removed in vacuo. The residue was taken in ethyl acetate (200 mL), washed with water (2×150 mL), dried over sodium sulfate and filtered. The solvent was removed in vacuo and the crude product was purified by chromatography on silica (160 g) with 2.5% ethyl acetate in heptane (2 L) and 5% ethyl acetate in heptane (2 L) to afford 29 (4.28 g, 71%).

Synthesis of 2-(Propoxy-d₇)benzoic acid (30-d7). A solution of 29 (4.2 g, 20.9 mmol) in tetrahydrofuran (80 mL) and 10% sodium hydroxide (40 mL) was stirred at reflux for 8 h. Tetrahydrofuran was removed in vacuo and the aqueous residue was diluted with water (50 mL), washed with MTBE (2×100 mL), cooled on an ice bath and made acidic with 6N hydrochloric acid. The resulting mixture was extracted with ethyl acetate (2×60 mL), dried over sodium sulfate and filtered. The solvent was removed in vacuo and the residue was dried in a vacuum oven (60° C.) to give 30-d7 (3.79 g, 97%).

Synthesis of 1-(Methyl-d₃)-4-(2-(propoxy-d₇)benzamido)-3-(2,2,3,3,3-d₅-propyl)-1H-pyrazole-5-carboxamide (33-d15). To a solution of 30-d7 (0.94 g, 5 mmol) in dichloromethane (20 mL) was added thionyl chloride (2 g) and the resulting mixture was stirred at reflux for 2 h. Solvent and excess thionyl chloride were removed in vacuo. The crude acid chloride (31-d7) in dichloromethane (4 mL) was added at 0° C. to a suspension of 32-d8 (1.15 g, 5 mmol) in dichloromethane (25 mL). This mixture was stirred at 0° C. and diisopropylethylamine (0.78 g, 6 mmol) in dichloromethane (5 mL) was added at such a rate that the temperature did not exceed 5° C. Stirring was continued at 0° C. for 20 min and at rt for 2 h. The reaction mixture was diluted with dichloromethane (150 mL), washed with water (2×80 mL) and saturated sodium bicarbonate solution (100 mL), dried over sodium sulfate and filtered. The solvent was removed in vacuo. The residue was taken up in heptane (75 mL), stirred at 70° C. for 20 min and allowed to cool overnight. The precipitate was collected by filtration, washed with heptane and dried in a vacuum oven (60° C.) to give 33-d15 (1.46 g, 81%).

Synthesis of 3-(5-Carbamoyl-1-(methyl-d₃)-3-(2,2,3,3,3-d₅-propyl)-1H-pyrazol-4-ylcarbamoyl)-4-(propoxy-d₇)benzene-1-sulfonyl chloride (34-d15). To chlorosulfonic acid (5 mL) stirred on an ice bath under nitrogen was slowly added 33-d15 (1.4 g, 3.89 mmol) at such a rate that the temperature did not exceed 5° C. After addition was complete, stirring was continued on the ice bath for 15 min, then at rt overnight. The reaction mixture was poured on ice (20 g). Water was added (30 mL) and the resulting suspension was stirred at rt for 30 min. The precipitate was collected by filtration, washed with water and dried in a vacuum oven (60° C.) to afford 34-d15 (1.32 g, 74%).

Synthesis of 1-(Methyl-d₃)-4-(5-(N-(2-(1-methylpyrrolidin-2-yl)-1,1-d₂-ethyl)sulfamoyl)-2-(propoxy-d₇)benzamido)-3-(2,2,3,3,3-d₅-propyl)-1H-pyrazole-5-carboxamide (36-d17). A solution of 34-d15 (1.32 g, 2.88 mmol) in dichloromethane (25 mL) was stirred under nitrogen on an ice bath and 35-d2 (0.78 g, 6.05 mmol) was added. Stirring was continued at rt for 2 h. The reaction mixture was diluted with dichloromethane (to 150 mL), washed with water (100 mL), saturated sodium bicarbonate solution (100 mL) and brine (100 mL), dried over sodium sulfate and filtered. The solvent was removed in vacuo, the solid residue was dissolved in ethyl acetate (50 mL) at 60° C. (water bath), the volume was reduced to 10 mL and this solution was allowed to sit overnight. The precipitate was collected by filtration, washed with a little cold ethyl acetate and dried in a vacuum oven (60° C.) to give 36-d17 (0.93 g, 58.5%).

Synthesis of 3-(1-(Methyl-d₃)-7-oxo-3-(2,2,3,3,3-d₅-propyl)-6,7-dihydro-1H-pyrazolo[4,3-d]pyrimidin-5-yl)-N-(2-(1-methylpyrrolidin-2-yl)-1,1-d₂-ethyl)-4-(propoxy-d₇)-benzenesulfonamide (Compound 210). A solution of 36-d17 (0.90 g, 1.63 mmol) and potassium tert-butoxide (0.37 g, 3.3 mmol) in tert-butanol (15 mL) was stirred at reflux under nitrogen for 5 h. After cooling, the reaction mixture was diluted with ethyl acetate (80 mL), washed with water (2×60 mL) and brine (80 mL), dried over sodium sulfate and filtered. The solvent was removed in vacuo and the crude product was dissolved in hot ethyl acetate (50 mL, 60° C.), treated with charcoal, and filtered hot. The solvent was removed in vacuo and the product was dried in a vacuum oven (60° C.) to yield Compound 210 (0.65 g, 74.7%). ¹H-NMR (300 MHz, CDCl₃): δ 1.40-1.71 (m, 4H), 1.73-1.87 (m, 2H), 2.07-2.16 (m, 1H), 2.31 (s, 3H), 2.36-2.42 (m, 2H), 2.91 (s, 2H), 3.04-3.10 (m, 1H), 7.14 (d, J=8.8, 1H), 7.95 (dd, J₁=8.8, J₂=2.6, 1H), 8.94 (d, J=2.6, 1H), 10.85 (bs, 1H). ¹³C-NMR (75 MHz, CDCl₃): δ 22.86, 27.60, 28.08, 28.16, 40.91, 57.06, 65.07, 113.05, 121.20, 130.50, 131.43, 133.90, 146.88, 147.22, 153.84, 159.29. HPLC (method: 20 mm C18-RP column-gradient method 2-95% ACN+0.1% formic acid in 3.3 min with 1.7 min hold at 95% ACN; Wavelength: 254 nm): retention time: 2.72 min; 99.8% purity. MS (M+H): 534.3. Elemental Analysis (C₂₅H₁₉D₁₇N₆O₄S): Calculated: C=56.26, H=6.80, N=15.74, S=6.01. Found: C=56.01, H=6.69, N=15.47, S=6.01.

### Example 10

Determination of Metabolic Stability of Test Compounds using Human Liver Microsomes. The metabolic stability of compounds of the invention was tested using pooled liver microsomal incubations. Full scan LC-MS analysis was then performed to detect major metabolites. Samples of the test compounds, exposed to pooled human liver microsomes, were analyzed using HPLC-MS (or MS/MS) detection. For determining metabolic stability, multiple reaction monitoring (MRM) was used to measure the disappearance of the test compounds. For metabolite detection, Q1 full scans were used as survey scans to detect the major metabolites.

Experimental Procedures. Human liver microsomes (“HLM”; 20 mg/mL) were obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) were purchased from Sigma-Aldrich.

Stock solutions (7.5 mM) of Compounds 100, 103, 108 and 210 and udenafil were prepared in DMSO. The 7.5 mM stock solutions were diluted to 50 μM in acetonitrile (ACN). The 20 mg/mL human liver microsomes were diluted to 0.625 mg/mL in 0.1 M potassium phosphate buffer, pH 7.4, containing 3 mM MgCl₂. The diluted microsomes (375 μL) were added to wells of a 96-well deep-well polypropylene plate in triplicate. Ten μL of the 50 μM test compound solution was added to the microsomes and the mixture was pre-warmed for 10 minutes. Reactions were initiated by addition of 125 μL of pre-warmed NADPH solution (8 mM NADPH in 0.1M potassium phosphate buffer, pH 7.4, containing 3 mM MgCl₂). The final reaction volume was 0.5 mL and contained 0.5 mg/mL human liver microsomes, 1 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow-well 96-well plates which contained 50 mL of ice-cold ACN with internal standard to stop the reactions. The plates were stored at 4° C. for 20 minutes after which 100 mL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer.

The in vitro t_{1/2}s for test compounds were calculated from the slopes of the linear regression of % parent remaining (ln) vs incubation time relationship using the following formula:

in vitro t_{1/2}=0.693/k, where k=−[slope of linear regression of % parent remaining(ln) vs incubation time]. Data analysis was performed using Microsoft Excel Software.

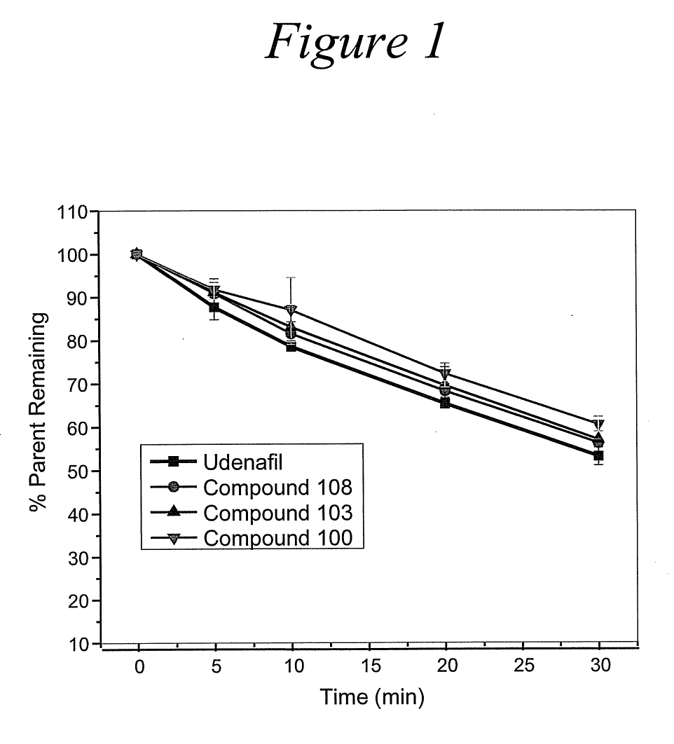

The results for Compounds 100, 103, and 108 are depicted in FIG. 1. The experiment was run an additional three times using all four of Compounds 100, 103, 108, and 210 and udenafil and the results are tabulated in Table 2, below.

TABLE 2
Metabolic Stability of Compounds of the Invention

| Compound | Avg t_{1/2} (min) ± SD |
|---|---|
| Udenafil | 33.6 ± 2.2 |
| 100 | 41.3 ± 1.5 |
| 103 | 37.2 ± 1.8 |
| 108 | 36.2 ± 1.5 |
| 210 | 42.4 ± 3.74 |

Under the assay conditions tested (0.5 mg/mL, 1 mM test compd) the t_{1/2}s of Compounds 103 and 108 showed a slight (≦10%) differentiation as compared to udenafil. Compounds 100 and 210 demonstrated t_{1/2}s that were ˜20% longer compared to udenafil.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

What is claimed is:

1. A compound of Formula A: 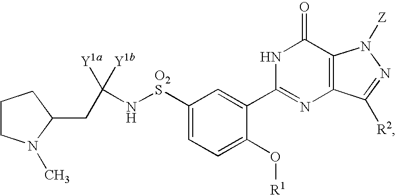
or a pharmaceutically acceptable salt thereof, wherein:
each Y¹ is independently selected from hydrogen and deuterium;
Z is selected from —CH₃, —CH₂D, —CHD₂ and —CD₃;
R¹ and R² are each independently n-propyl optionally substituted with deuterium; and
at least one of R¹, R² and Z comprises a deuterium atom, or at least one Y is deuterium.

2. The compound of claim 1, wherein each of R¹ and R² is —(CH₂)₂CH₃, the compound having the formula (I): 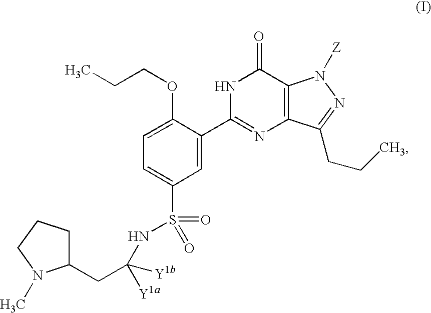
or a pharmaceutically acceptable salt thereof.

3. The compound of claim 2, wherein:
Y^{1a} and Y^{1b} are the same; and
Z is selected from —CH₃ and —CD₃.

4. The compound of claim 1, wherein:
Y^{1a} and Y^{1b} are the same;
R¹ is selected from —CH₂CH₂CH₃, and —CD₂CD₂CD₃;
R² is selected from —CH₂CH₂CH₃, —CH₂CD₂CD₃, and —CD₂CD₂CD₃; and
Z is selected from —CH₃ and —CD₃.

5. The compound of claim 1, selected from any one of the compounds set forth in the table below:

| Compound | Y^{1a} | Y^{1b} | Z | R¹ | R² |
|---|---|---|---|---|---|
| 100 | D | D | CD₃ | —CH₂CH₂CH₃ | —CH₂CH₂CH₃ |
| 101 | D | D | CH₂D | —CH₂CH₂CH₃ | —CH₂CH₂CH₃ |
| 102 | D | D | CHD₂ | —CH₂CH₂CH₃ | —CH₂CH₂CH₃ |
| 103 | D | D | CH₃ | —CH₂CH₂CH₃ | —CH₂CH₂CH₃ |
| 104 | D | H | CD₃ | —CH₂CH₂CH₃ | —CH₂CH₂CH₃ |
| 105 | D | H | CH₂D | —CH₂CH₂CH₃ | —CH₂CH₂CH₃ |
| 106 | D | H | CHD₂ | —CH₂CH₂CH₃ | —CH₂CH₂CH₃ |
| 107 | D | H | CH₃ | —CH₂CH₂CH₃ | —CH₂CH₂CH₃ |
| 108 | H | H | CD₃ | —CH₂CH₂CH₃ | —CH₂CH₂CH₃ |
| 109 | H | H | CH₂D | —CH₂CH₂CH₃ | —CH₂CH₂CH₃ |
| 110 | H | H | CHD₂ | —CH₂CH₂CH₃ | —CH₂CH₂CH₃ |
| 200 | H | H | CH₃ | —CH₂CH₂CH₃ | —CH₂CD₂CD₃ |
| 201 | H | H | CH₃ | —CD₂CD₂CD₃ | —CH₂CD₂CD₃ |
| 202 | H | H | CH₃ | —CD₂CD₂CD₃ | —CH₂CH₂CH₃ |
| 203 | H | H | CD₃ | —CH₂CH₂CH₃ | —CH₂CD₂CD₃ |
| 204 | H | H | CD₃ | —CD₂CD₂CD₃ | —CH₂CD₂CD₃ |
| 205 | H | H | CD₃ | —CD₂CD₂CD₃ | —CH₂CH₂CH₃ |
| 206 | D | D | CH₃ | —CH₂CH₂CH₃ | —CH₂CD₂CD₃ |
| 207 | D | D | CH₃ | —CD₂CD₂CD₃ | —CH₂CD₂CD₃ |
| 208 | D | D | CH₃ | —CD₂CD₂CD₃ | —CH₂CH₂CH₃ |
| 209 | D | D | CD₃ | —CH₂CH₂CH₃ | —CH₂CD₂CD₃ |
| 210 | D | D | CD₃ | —CD₂CD₂CD₃ | —CH₂CD₂CD₃ |
| 211 | D | D | CD₃ | —CD₂CD₂CD₃ | —CH₂CH₂CH₃ |

6. The compound of claim 5, selected from any one of Compound 100, Compound 103, Compound 108 and Compound 210.

7. The compound of claim 1, wherein any atom not designated as deuterium in any of the embodiments set forth above is present at its natural isotopic abundance.

8. A pyrogen-free pharmaceutical composition comprising:
a compound of claim 1, or a pharmaceutically acceptable salt thereof; and
a pharmaceutically acceptable carrier.

9. The composition of claim 8 additionally comprising a second therapeutic agent useful in the treatment or prevention of a disease or condition selected from erectile dysfunction, benign prostatic hyperplasia, and pulmonary arterial hypertension.

10. A method of treating a human patient suffering from or susceptible to a disease or condition selected from erectile dysfunction, benign prostatic hyperplasia, and pulmonary arterial hypertension, comprising the step of administering to the human patient in need thereof a composition of claim 8.

11. The method of claim 10, wherein the patient is suffering from or susceptible to erectile dysfunction.

12. A method of treating a patient suffering from or susceptible to dysmenorrhea comprising the step of co-administering to the patient in need thereof a composition of claim 8 and a vasopressin receptor antagonist.
